(19) United States
(12) Patent Application Publication
(10) Pub. No.: US 2026/0257976 A1
(43) Pub. Date: Sep. 3, 2026

(54) PROCESS FOR PURIFYING HYDROCARBON STREAMS INCLUDING HETEROGENEOUSLY AND HOMOGENEOUSLY CATALYSED REACTIONS

(71) Applicant: Evonik Oxeno GmbH & Co. KG, Marl (DE)

(72) Inventors: Stephan Peitz, Oer-Erkenschwick (DE); Guido Stochniol, Haltern am See (DE); Martin Althoff, Marl (DE); Ralf Boll, Dorsten (DE); Helena Lopez-Fernandez, Herne (DE)

(73) Assignee: Evonik Oxeno GmbH & Co. KG, Marl (DE)

(21) Appl. No.: 18/866,253

(22) PCT Filed: Apr. 27, 2023
(86) PCT No.: PCT/EP2023/061158

§ 371 Date: Nov. 15, 2024

(30) Foreign Application Priority Data
May 18, 2022 (EP) 22173976.6

(51) Int. Cl. C07C 7/00; C07C 7/04; C07C 7/148; C07C 7/163; C07C 7/177

(52) U.S. Cl. CPC C07C 7/005; C07C 7/04; C07C 7/14841; C07C 7/163; C07C 7/177; C07C 2523/46; C07C 2523/75; C07C 2523/755

(57) ABSTRACT

A process purifies a hydrocarbon stream having at least Cₓ alkanes, Cₓ olefins, low boilers such as Cₓ₋₁ hydrocarbons and high boilers such as Cₓ₊₁ hydrocarbons, with x=3 to 5. The process involves carrying out first a low boiler removal and then a separation into two partial streams which are each subjected to a chemical reaction. Once the products have been removed, the streams are combined again and subjected to a high boiler removal.

The invention provides a process for purifying a hydrocarbon stream containing at least Cₓ alkanes, Cₓ olefins, low boilers such as Cₓ₋₁ hydrocarbons and high boilers such as Cₓ₊₁ hydrocarbons, with x=3 to 5. The process according to the invention involves carrying out first a low boiler removal and then a separation into two partial streams which are each subjected to a chemical reaction. Once the products have been removed, the streams are combined again and subjected to a high boiler removal.

The provision of C3 to C5 alkane streams which can be used for example as propellant gases has long been known. In order to be able to be used as propellant gases, the C3 to C5 alkane streams obtained need to exhibit certain product specifications, i.e. may contain only small amounts of unwanted and/or odour-forming substances. The term “odour-forming substance” in this connection is any substance or compound not corresponding to the substance that constitutes the propellant gas. If the propellant gases are to be used in the cosmetics or medical field, even any contamination with reactive components such as olefins, but also any odour contamination with other substances or compounds, should be avoided since applications close to the body are usually involved.

The general object is that of improving the known processes in order to obtain streams in which there is less contamination with foreign odour-forming substances. It is also important here to provide a process which is as efficient and cost-effective as possible.

It has surprisingly been found that the separation of a stream which has been obtained from a low boiler removal and the passage through different reaction steps results in lower contamination with foreign odour-forming substances.

The process according to the invention is accordingly a process for purifying a hydrocarbon stream containing at least Cₓ alkanes, Cₓ olefins, low boilers such as Cₓ₋₁ hydrocarbons and high boilers such as Cₓ₊₁ hydrocarbons, where x is an integer from 3 to 5, wherein the process comprises the following steps:
a) removing at least a portion of the low boilers from the hydrocarbon stream in a first removal unit to obtain a hydrocarbon stream which has been at least partially freed of low boilers, and separating the hydrocarbon stream which has been at least partially freed of low boilers into two partial streams A1 and A2;
b) carrying out a heterogeneously catalysed reaction with the partial stream A1 using a nickel-containing catalyst to obtain a reaction mixture A1;
c) removing the obtained reaction product from the reaction mixture A1 to obtain a hydrocarbon stream A1 from which the reaction products from step b) have been at least partially removed;
d) carrying out a homogeneously catalysed reaction with the partial stream A2 to obtain a reaction mixture A2;
e) removing the obtained reaction product from the reaction mixture A2 to obtain a hydrocarbon stream A2 from which the reaction products from step d) have been at least partially removed;
f) feeding the hydrocarbon stream A1 from step c) and the hydrocarbon stream A2 from step e) to a hydrogenation to obtain a hydrogenation product;
g) removing at least a portion of the high boilers from the hydrogenation product from step f) in a second removal unit to obtain a purified hydrocarbon stream containing more than 98% by weight of Cx alkanes.

The process according to the invention has the advantage that a high-purity alkane stream may thereby be obtained. Both a homogeneously catalysed and a heterogeneously catalysed reaction are operated at the same time, as a result of which the olefins contained in the hydrocarbon stream used can be converted into more valuable products. This makes the separation task in the high boiler removal in the last stage easier to accomplish. In addition, it is possible to respond flexibly to market requirements. If products from the heterogeneously catalysed reaction are more in demand, the partial amount A1 of the hydrocarbon stream which has been freed of low boilers and which is guided to the heterogeneously catalysed reaction can be increased in order to produce more of these products. The same also applies for the opposite case if the demand for the product of the homogeneously catalysed reaction is greater, the partial amount A2 then being increased. A further advantage of the present process is that it can be integrated into existing petrochemical production plants.

The hydrocarbon stream to be purified is a Cₓ hydrocarbon stream with x=3 to 5, i.e. x=3, 4 or 5. Such streams contain both Cₓ alkanes and Cₓ olefins, but also low boilers such as Cₓ₋₁ hydrocarbons and high boilers such as Cₓ₊₁ hydrocarbons. Such streams are available on an industrial scale from cracking processes and are used in a wide variety of petrochemical processes. The streams used here do not contain any alkyl tert-butyl ethers (ATBE) such as MTBE (methyl tert-butyl ethers) or ETBE (ethyl tert-butyl ethers), since these would enter into the reaction steps described below as high boilers and could lead to serious problems there. The aim of the present invention is to obtain streams containing more than 98% by weight of Cₓ alkanes, since there is a need for such streams. To this end, the streams are purified according to the invention.

The hydrocarbon stream to be purified is accordingly a C3 hydrocarbon stream, a C4 hydrocarbon stream, or a C5 hydrocarbon stream. C3 hydrocarbon streams according to the invention contain at least propene, propane, C2 hydrocarbons (e.g. ethene, ethane) and C4 hydrocarbons (e.g. butane, butene). Such a C3 hydrocarbon stream is also referred to in the context of the present invention as propene stream (to be purified). C4 hydrocarbon streams according to the invention contain at least butene (e.g. 1-butene, 2-butene, possibly isobutene), butane, C 3 hydrocarbons (e.g. propene, propane) and C5 hydrocarbons (e.g. pentene, pentane). Such a C4 hydrocarbon stream is also referred to in the context of the present invention as butene stream or isobutene stream (to be purified). C5 hydrocarbon streams according to the invention contain at least pentene (e.g. 1-pentene, cis- or trans-2-pentene), pentane, C4 hydrocarbons (e.g. butene, butane) and C6 hydrocarbons (e.g. hexene, hexane). Such a C5 hydrocarbon stream is also referred to in the context of the present invention as pentene stream (to be purified). In a preferred embodiment, a butene stream is used as hydrocarbon stream to be purified.

The composition of the butene stream preferably used is fundamentally not limited to a particular composition. The only prerequisite is that the individual components according to Claim 1 are present. Preferred butene streams contain butane, isobutane, butene (1-butene and 2-butene and possibly isobutene). According to the invention, however, the butene stream may contain from 0.0001% up to 3% by weight of C3 hydrocarbons, from 0.01% up to 90% by weight of butene and from 0.0001% up to 20% by weight of C5 hydrocarbons. In addition, the butene stream may additionally contain, inter alia, small amounts of dimethyl ether, water, methanethiol, ethanethiol, dimethyl sulfide, dimethyl disulfide and/or hydrogen sulfide (in each case up to 100 ppm by weight). The butene stream may contain further substances or compounds in traces, which cannot be enumerated individually. Some of these substances may have a perceptible odour. The presence thereof is unwanted and is minimized by the process described here.

In the first step a) of the process according to the invention, the hydrocarbon stream to be purified, especially the propene stream, the butene stream or the pentene stream, is fed to a low boiler removal in a first removal unit where at least a portion of the low boilers are removed. Suitable processes are fundamentally familiar to those skilled in the art. The removal of at least a portion of the low boilers is preferably carried out in one or more distillation columns, particularly preferably in a single distillation column. It will be apparent that the low boilers present are obtained at the top and are at least partially removed there. The hydrocarbon stream which has been at least partially freed of low boilers by way of the low boiler removal in step a) may then in particular be removed as bottom stream.

The distillation column preferably used for the removal of at least a portion of the low boilers in step a) may in principle be configured as desired and have any desired fittings such as separation stages, provided that the function, i.e. the removal of at least a portion of low boilers, is ensured.

In a further preferred embodiment of the present invention, the removal of at least a portion of the low boilers in step a) is carried out at a pressure of 1 to 25 barg, particularly preferably 2 to 20 barg. The temperature in the removal in step a) is preferably in the range from 30° C. to 150° C., particularly preferably in the range from 35° C. to 100° C. In the context of the present invention, the unit barg means bar gauge pressure, i.e. a gauge pressure above the prevailing ambient pressure.

Once at least a portion of the low boilers have been removed from the hydrocarbon stream used, the hydrocarbon stream obtained which has been at least partially freed of low boilers is separated into two partial streams A1 and A2. This separation is also part of step a) in the context of the present invention. By way of example, such a separation may be effected in a simple manner using a T-piece in the line together with one or more valve(s).

The partial stream A1 is then subjected to a heterogeneously catalysed reaction in step b). This uses a nickel-containing catalyst. The olefins contained in the partial stream A1 are at least partially converted by the heterogeneously catalysed reaction. A reaction mixture A1 is thus obtained which contains at least the reaction products formed in the reaction, unconverted olefins and the Cx alkanes. The heterogeneously catalysed reaction which is preferred in the context of the present invention and may be carried out as step b) of the process according to the invention is an oligomerization of the contained olefins, i.e. propene, butene or pentene.

In a preferred embodiment of the present invention, the heterogeneously catalysed reaction in step b) is an oligomerization. The olefins contained in the partial stream A1, Le. propene, butene or pentene, are converted here into the corresponding oligomers. Since it is particularly preferred in the context of the present invention to use a butene stream in step a), this would result in an oligomerization of butenes in step b).

The heterogeneous nickel-containing catalyst used in the oligomerization may be selected from the known nickel catalysts suitable for use in the oligomerization. Numerous catalyst systems are known to those skilled in the art. By way of example, reference is made here to the catalysts whose production has been described in EP 21213152.8, EP 3 546 065 A1 and EP 3 549 669A1. In general, the catalyst systems disclosed therein have a composition of 15% to 40% by weight, preferably 15% to 30% by weight, of NIO, 5% to 30% by weight of Al₂O₃, 55% to 80% by weight of SiO₂ and up to 2.5% by weight, preferably 0.01% to 2% by weight, of an alkali metal oxide, preferably sodium oxide. The figures are each based on a total composition of 100% by weight.

According to the invention, the nickel-containing catalyst used in step b) for the oligomerization may have a specific surface area (calculated according to BET) of 150 to 400 m²/g, preferably 190 to 350 m²/g, particularly preferably of 220 to 330 m²/g. The BET surface area is measured by nitrogen physisorption according to DIN ISO 9277 (version: 2014-01).

In a further preferred embodiment, the nickel-containing catalyst used in step b) for the oligomerization comprises mesopores and macropores, i.e. has a bimodal pore size distribution. The mesopores of the nickel-containing catalyst used in step b) for the oligomerization have an average pore diameter of 5 to 15 nm, preferably of 7 to 14 nm, particularly preferably of 9 to 13 nm. In contrast, the macropores of the nickel-containing catalyst used in step b) for the oligomerization preferably have an average pore diameter of 1 to 100 μm, particularly preferably of 2 to 50 μm. The average pore volume of the nickel-containing catalyst used in step b) for the oligomerization, i.e. of both the mesopores and the macropores, may be 0.5 to 1.5 cm³/g, preferably 0.7 to 1.3 cm³/g. The average pore diameter and the average pore volume may be determined by mercury porosimetry according to DIN 66133 (version: 1993-06).

Furthermore, the nickel-containing catalyst used in step b) for the oligomerization may have an average particle diameter (d50) of 0.1 mm to 7 mm, preferably 0.5 to 6 mm, particularly preferably of 1 mm to 5 mm. The average particle diameter may be determined by imaging methods, in particular determined by those in the standards ISO 13322-1 (version: 2004-12-01) and ISO 13322-2 (version: 2006-11-01). A suitable device for analysing the particle diameter is for example the Camsizer 2006 (Retsch Technology).

If an oligomerization is carried out in step b), it may be carried out under conditions known to those skilled in the art. In the context of the present invention, the oligomerization in step b) is preferably effected at a temperature in the range from 50° C. to 200° C., further preferably 60° C. to 180° C., particularly preferably in the range from 60° C. to 130° C. The pressure may be in the range from 10 to 70 bar, preferably in the range from 20 to 55 bar, in the oligomerization in step b). If the oligomerization is to be effected in the liquid phase, the parameters of pressure and temperature must to this end be chosen such that the reactant stream (the olefins or olefin mixtures used) is in the liquid phase. The weight-based space velocities (reactant mass per unit catalyst mass per unit time; weight hourly space velocity (WHSV)) are in the range between 1 g of reactant per g of catalyst per h (=1 h⁻¹) and 190 h⁻¹, preferably between 2 h⁻¹ and 35 h⁻¹, particularly preferably between 3 h⁻¹ and 25 h⁻¹.

The reaction mixture A1 obtained from the heterogeneously catalysed reaction in step b) is then worked up in step c) by at least partially removing the reaction products from step b). A hydrocarbon stream A1 is obtained here which is depleted of the reaction products from step b). The removal of the reaction products in step c) is preferably effected by means of distillation. The conditions of the distillation, i.e. temperature and pressure for example, are usually determined by the set-up (column height, number of trays, type of trays/packing, spacings etc.). The separation properties of the distillation may also be controlled during operation via the temperature distribution and/or the heat supply into the column and the reflux in the distillate. The separation property may also be adjusted within a certain range by changing the pressure. The precise settings therefore cannot be defined superordinately to and independently of the set-up of the distillation column, which however is known to those skilled in the art.

The hydrocarbon stream A1 obtained from step c) and from which the reaction products from step b) have been at least partially removed is guided to the hydrogenation in step f). This has the advantage that even small residues of olefins are hydrogenated and a hydrocarbon stream that is as pure as possible can thus be obtained after the removal in step g). The hydrogenation is the last reaction step in the process according to the invention, after which the hydrogenated stream is only subjected to a high boiler removal.

The hydrogenation is preferably a liquid phase hydrogenation. The process conditions, such as the pressure, should be chosen such that a liquid phase hydrogenation are possible. Appropriate conditions are familiar to those skilled in the art. In the hydrogenation in step f), olefins still contained in the hydrocarbon stream A1 from step c) are hydrogenated as completely as possible to form the corresponding alkanes. The hydrogenation in step f) is preferably carried out in a hydrogenation unit, consisting of one or more reactors. The reactors may be operated in circulation or in a straight pass. In a preferred embodiment of the present invention, the hydrogenation is carried out in at least two reactors, the first reactor being operated in circulation and the second and possible further reactors being operated in a straight pass. Hydrogen is used in the hydrogenation preferably in a slight stoichiometric excess, particularly preferably in a stoichiometric excess of 5% to 30%.

Known supported catalysts which at least one transition metal from the group consisting of palladium, platinum, rhodium, ruthenium, nickel or mixtures thereof and a supporting material from the group consisting of aluminium oxide, silicon dioxide, titanium dioxide, magnesium oxide or mixtures thereof may be used in the hydrogenation in step f). Alternatively, it is possible in principle to use carbon-based supports, for example activated carbon, graphitic supports, carbon nanotubes or the like. In a preferred embodiment of the present invention, use is made in the hydrogenation in the optional step of a supported catalyst which contains palladium or platinum as transition metal and aluminium oxide as supporting material.

The hydrogenation in step f) is preferably carried out at a temperature of 25° C. to 80° C., particularly preferably at a temperature of 35° C. to 60° C. The pressure is preferably 5 to 25 barg, particularly preferably 8 to 15 barg, in the hydrogenation in step f). These conditions apply in particular if a butene stream is used in the process according to the invention. Following the hydrogenation f) is a phase separation, known to those skilled in the art, in order to separate the gas phase, which comprises unconverted hydrogen and possibly also small amounts of hydrocarbons, from the liquid phase, which is then fed to the removal in step g).

With the exception of the hydrogenation in step f), this order of the process steps described above relates exclusively to the partial stream A1 obtained from step a). The partial stream A2 from step a) is instead subjected to a homogeneously catalysed reaction in step d). The homogeneously catalysed reaction may be a hydroformylation, an alkoxycarbonylation or an epoxidation.

The epoxidation may be carried out by means of processes familiar to those skilled in the art. Conceivable epoxidizing agents are all active oxygen-containing substances, for example hydrogen peroxide, peracetic acid or other peracids or else oxygen. The epoxidation may be carried out either in the gas phase with heterogeneous catalysts or else in the liquid phase with heterogeneous or homogeneous catalysts. Suitable processes for the epoxidation are described in the prior art for example in WO 2011/107199 A2 or WO 2017/089075 A1.

If the homogeneously catalysed reaction in step d) is a hydroformylation, the following process conditions are preferred:

The olefins used in the process are hydroformylated with syngas in the presence of a homogeneously dissolved catalyst system. The molar ratio between syngas and the feedstock mixture should be between 6:1 and 1:1, preferably between 3:1 and 1:1, particularly preferably between 2:1 and 1:1. The hydroformylation may optionally be carried out in the presence of a solvent known to those skilled in the art.

The homogeneous catalyst system usable in the hydroformylation may comprise Co or Rh, preferably Rh, and preferably a phosphorus-containing ligand. Appropriate catalyst systems are familiar to those skilled in the art. In principle, Co-based hydroformylations can also be carried out without ligands. In a particularly preferred embodiment, the homogeneous catalyst system comprises or consists of Rh and a phosphorus-containing ligand. Suitable ligands for the catalyst systems according to the invention are known to those skilled in the art (see e.g. the textbooks “Rhodium Catalyzed Hydroformylation” (from 2002) by P. W. N. M. van Leeuwen or “Hydroformylation—Fundamentals, Processes and Applications in Organic Synthesis” (from 2016) by A. Börner and R. Franke).

If Rh is used, the phosphorus-containing ligand for the catalyst system according to the invention is preferably a phosphine (e.g. TPP (triphenylphosphine)), a monophosphite (e.g. Alkanox 240 (tris(2,4-di-tert-butylphenyl)phosphite)) or a bisphosphite (e.g. BiPhePhos). It is also possible to use mixtures of ligands.

The temperature in the homogeneously catalysed hydroformylation is preferably in the range from 80° C. to 250° C., further preferably in the range from 90° C. to 225° C. and particularly preferably in the range from 100° C. to 210° C. The pressure in the homogeneously catalysed hydroformylation is preferably in the range from 20 to 350 bar, further preferably in the range from 30 to 325 bar and particularly preferably in the range from 45 to 300 bar.

The pressure in the hydroformylation usually corresponds to the total gas pressure. In the context of the present invention, the total gas pressure means the sum of the pressures occurring of all gaseous substances present, i.e. the pressure of the (total) gas phase. In the present process, this corresponds in particular to the sum of the partial pressures of CO and H2, i.e. the total gas pressure is then the syngas pressure.

Homogeneously catalysed hydroformylations may be operated as liquid discharge processes (“liquid recycle”) or as gas discharge processes (“gas recycle”). Both process variants are known to those skilled in the art and described in numerous textbooks. A specific selection of such a process is not necessary in the context of the present invention because the process can fundamentally be carried out in both ways.

If the homogeneously catalysed reaction in step d) is an alkoxycarbonylation, the following process conditions are preferred:

The homogeneous catalyst system used for the alkoxycarbonylation preferably comprises at least one metal from group 8 to 10 of the Periodic Table of the Elements (PTE) or a compound thereof, a phosphorus-containing ligand and an acid as cocatalyst.

The metal from group 8 to 10 of the PTE is preferably palladium. The palladium is preferably used in the form of a precursor compound as a palladium compound coordinated by the phosphorus-containing ligand. Examples of palladium compounds that may be used as precursor compounds are palladium chloride [PdCl₂], palladium(II) acetylacetonate [Pd(acac)₂], palladium(II) acetate [Pd(OAc)₂], dichloro(1,5-cyclooctadiene)palladium(II) [Pd(cod)₂Cl₂], bis(dibenzylideneacetone)palladium(0) [Pd(dba)₂], tris(dibenzylideneacetone)dipalladium(0) [Pd₂(dba)₃], bis(acetonitrile)dichloropalladium(II) [Pd(CH₃CN)₂Cl₂], palladium(cinnamyl)dichloride [Pd(cinnamyl)Cl₂]. Preference is given to using the compounds [Pd(acac)₂] or [Pd(OAc)₂]. The concentration of palladium metal in the alkoxycarbonylation is preferably between 0.01 and 0.6 mol %, preferably between 0.03 and 0.3 mol %, particularly preferably between 0.04 and 0.2 mol %, based on the molar amount of the hydrocarbon used.

Suitable phosphorus-containing ligands of the catalyst system according to the invention preferably have a bidentate structure. Preferred phosphorus-containing ligands for the catalyst system according to the invention are benzene-based diphosphine compounds, as disclosed for example in EP 3 121 184A2. The ligands may be combined with the palladium in a preliminary reaction so that the palladium-ligand complex is fed into the reaction zone, or added to the reaction in situ and combined with the palladium there. The molar ratio of ligand:metal may be 1:1 to 10:1, preferably 2:1 to 6:1, particularly preferably 3:1 to 5:1, in the alkoxycarbonylation.

In the alkoxycarbonylation, the homogeneous catalyst system further comprises an acid, where it may in particular be a Brønsted or a Lewis acid. The Lewis acid used may in particular be aluminium triflate, aluminium chloride, aluminium hydride, trimethylaluminium, tris(pentafluorophenyl)borane, boron trifluoride, boron trichloride or mixtures thereof. Of the Lewis acids mentioned, preference is given to using aluminium triflate. The Lewis acid is preferably added in a molar ratio of Lewis acid:ligand of 1:1 to 20:1, preferably 2:1 to 15:1, particularly preferably 5:1 to 10:1.

Suitable Brønsted acids preferably have an acid strength of pKa≤5, particularly preferably an acid strength of pKa≤3. The stated acid strength pKa relates to the pKa determined under normal conditions (25° C., 1.01325 bar). For a polyprotic acid, the acid strength pKa relates in the context of this invention to the pKa of the first protolysis step. The Brønsted acid is preferably added in a molar ratio of Brønsted acid:ligand of 1:1 to 15:1, preferably 2:1 to 10:1, particularly preferably 3:1 to 5:1.

The Brønsted acid used may in particular be perchloric acid, sulfuric acid, phosphoric acid, methylphosphonic acid or sulfonic acids. Suitable sulfonic acids are for example methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid and dodecylsulfonic acid. Particularly preferred acids are sulfuric acid, methanesulfonic acid, trifluoromethanesulfonic acid and p-toluenesulfonic acid. The acid is preferably sulfuric acid. Carboxylic acids, on the other hand, are less suitable or not suitable at all.

The homogeneously catalysed alkoxycarbonylation is preferably carried out at a temperature of 25° C. to 140° C., further preferably at a temperature of 80° C. to 120° C. and particularly preferably at a temperature of 90° C. to 110° C. The pressure may be between 5 and 60 bar, preferably between 10 and 40 bar, particularly preferably between 15 and 30 bar.

The homogeneously catalysed alkoxycarbonylation affords a product mixture comprising at least the ester formed by the reaction, the homogeneous catalyst system, low boilers, for example low-boiling by-products such as ethers, high boilers, unreacted alcohols and any unreacted hydrocarbons. The product mixture may therefore be subjected to a subsequent catalyst removal. This may for example be effected with a membrane separation, whereby the homogeneous catalyst system and unreacted hydrocarbon and/or unreacted alcohol are enriched in the retentate, while the ester formed is enriched in the permeate. The retentate comprising the enriched homogeneous catalyst system may then be recycled into the reaction zone.

The reaction mixture A2 obtained from step d) which generally which contains at least the reaction products formed in the reaction, unconverted olefins and the Cx alkanes is then worked up in step e) by at least partially removing the reaction products from step d). A hydrocarbon stream A2 is obtained here which is depleted of the reaction products from step d). The removal of the reaction products from step d) is preferably effected by means of distillation. In the case of the homogeneously catalysed reactions, the catalyst system is also present in the reaction output and should be removed before the product removal. This may for example be effected by way of an evaporation and/or a membrane separation. Appropriate processes and conditions are familiar to those skilled in the art.

The hydrocarbon stream A2 obtained from step e) and from which the reaction products from step d) have been at least partially removed is then guided, together with the hydrocarbon stream A1 from step c), to the hydrogenation in step f). The process conditions have already been described above. The hydrogenation product obtained here is then fed to the removal in step g).

The intention of the removal in step g) is to remove high boilers that are still present in order to obtain a purified hydrocarbon stream containing more than 98% by weight of Cx alkanes. The purified hydrocarbon stream obtained containing more than 98% by weight of Cx alkanes is then discharged from the process. The discharged hydrocarbon stream can then be sold and used for example as propellant gas. Possible high boilers that may be removed here are the reaction products from steps c) and/or e) which may not have been removed completely in the removal operations following said steps.

The removal in step g) is preferably effected by means of distillation in one or more distillation columns, preferably in a single distillation column. The high boilers are obtained here in the bottom of the distillation column and the purified hydrocarbon stream is obtained at the top of the distillation column. The process conditions may vary depending on the composition and type of the hydrocarbon stream. However, the selection of suitable conditions does not pose any major problems to those skilled in the art. The removal of at least a portion of the high boilers in step g) may preferably be carried out at a pressure of 1 to 25 barg, particularly preferably 2 to 20 barg. The temperature in the removal in step g) is preferably in the range from 30° C. to 150° C., particularly preferably in the range from 35° C. to 100° C. This applies in particular if a butene stream is used in the process according to the invention.

1. A process for purifying a hydrocarbon stream containing at least Cx alkanes, Cx olefins, low boilers and high boilers, where x is an integer from 3 to 5, the process comprising:
a) removing at least a portion of the low boilers from the hydrocarbon stream in a first removal unit to obtain a hydrocarbon stream which has been at least partially freed of low boilers, and separating the hydrocarbon stream which has been at least partially freed of low boilers into two partial streams A1 and A2;
b) carrying out a heterogeneously catalyzed oligomerization with the partial stream A1 using a nickel-containing catalyst to obtain a reaction mixture A1;
c) removing an obtained reaction product from the reaction mixture A1 to obtain a hydrocarbon stream A1 from which reaction products from b) have been at least partially removed;
d) carrying out a homogeneously catalyzed reaction with the partial stream A2 to obtain a reaction mixture A2, wherein the homogeneously catalyzed reaction is a hydroformylation, an alkoxycarbonylation or an epoxidation;
e) removing an obtained reaction product from the reaction mixture A2 to obtain a hydrocarbon stream A2 from which reaction products from d) have been at least partially removed;
f) feeding the hydrocarbon stream A1 from c) and the hydrocarbon stream A2 from e) to a hydrogenation to obtain a hydrogenation product;
g) removing at least a portion of the high boilers from the hydrogenation product from f) in a second removal unit to obtain a purified hydrocarbon stream containing more than 98% by weight of Cx alkanes.

2. The process according to claim 1, wherein the hydrocarbon stream used and to be purified is a C4 hydrocarbon stream.

3. The process according to claim 1, wherein the removing in a) is carried out at a pressure of 2 to 10 barg.

4. The process according to claim 1, wherein the removal in a) is carried out at a temperature in a range from 30° C. to 90° C.

5. The process according to claim 1, wherein a heterogeneous nickel-containing catalyst is used in the heterogeneously catalyzed oligomerization in b).

6. The process according to claim 1, wherein the removing in c) is a distillation.

7. The process according to claim 1, wherein a homogeneous catalyst system in the hydroformylation comprises cobalt, Co, or rhodium, Rh.

8. The process according to claim 1, wherein a homogeneous catalyst system in the alkoxy carbonylation comprises a metal from group 8 to 10 of the Periodic Table of the Elements (PTE) or a compound thereof, a phosphorus-containing ligand and an acid as cocatalyst.

9. The process according to claim 1, wherein the removing in e) is a distillation.

10. The process according to claim 1, wherein the hydrogenation in f) is a liquid phase hydrogenation.

11. The process according to claim 10, wherein a supported catalyst comprising at least one transition metal selected from the group consisting of palladium, platinum, rhodium, ruthenium, nickel and mixtures thereof and a supporting material selected from the group consisting of aluminium oxide, silicon dioxide, titanium dioxide, magnesium oxide and mixtures thereof is used in the hydrogenation.

12. The process according to claim 1, wherein the removing in g) is carried out at a pressure in a range from 1 to 25 barg.

13. The process according to claim 1, wherein a purified hydrocarbon stream obtained in f) containing more than 98% by weight of Cx alkanes is discharged from the process.

14. The process according to claim 1, wherein the low boilers comprise Cx−1 hydrocarbons.

15. The process according to claim 1, wherein the high boilers comprise Cx+1 hydrocarbons.
